(19) United States
(12) Patent Application Publication
(10) Pub. No.: US 2014/0023712 A1
(43) Pub. Date: Jan. 23, 2014

(54) Aqueous Transparent Oil-In-Water Emulsion Comprising an Emulsified Carotenoid

(71) Applicant: BASF SE, Ludwigshafen (DE)

(72) Inventors: Thrandur Helgason, Ludwigshafen (DE); Christian Köpsel, Weinheim (DE); Clemens Sambale, Bohl-lggelheim (DE); Andreas Habich, Speyer (DE); Andreas Hasse, Weisenheim/Sand (DE)

(21) Appl. No.: 13/945,438

(22) Filed: Jul. 18, 2013

Related U.S. Application Data
(60) Provisional application No. 61/673,786, filed on Jul. 20, 2012.

(51) Int. Cl. A23L 1/275; A61K 47/08

(52) U.S. Cl. CPC A23L 1/2753; A61K 47/08
USPC 424/489; 426/540; 514/772

(57) ABSTRACT

The invention relates to an aqueous transparent oil-in-water emulsion comprising a carotenoid and a process for producing said emulsion.

The invention relates to an aqueous transparent oil-in-water emulsion comprising a carotenoid and a process for producing said emulsion.

Emulsions containing carotenoids are well known in the states of the art. Carotenoids are used as colouring material and as active substances for the human food and animal feed industry as well as in the pharmaceutical sector. In this area they are also used in the last years to replace synthetic azo dyes, otherwise known as the ‘Southampton Six’: sunset yellow (E110), quinoline yellow (E104), carmoisine (E122), allura red (E129), tartrazine (E102), and Ponceau 4R (E124). These might have negative effects, especially an adverse effect on activity and attention in children.

In the past it was difficult to replace the azo dyes by carotenoids and to maintain exactly the same colour match of the products as with azo dyes.

Carotenoids are insoluble in water. Additionally they show a great sensitivity to oxidation. Therefore it is difficult to use the carotenoids in crystalline form which is not stable during storage. Generally a lot of additives are necessary in order to obtain stable carotenoid emulsion, where the carotenoid is protected from oxidation and the emulsion have an improved colouration.

One approach to overcome the problems in handling carotenoids is the manufacturing of micro-emulsions. These micro-emulsions exhibit a soapy taste caused by the used emulsifiers.

Emulsifiers, especially ester of long-chain fatty acid with ascorbic acid, in particular ascorbyl palmitate, are also used according to the teaching of U.S. Pat. No. 4,844,934 to obtain an emulsion stable to creaming.

Formation of small emulsions, made by dispersion an liquid oil such as Vitamin E acetate into water phase with a emulsifier followed by homogenization at high pressure, requires high amounts of energy with high amount of emulsifier (these are common equipment used for emulsion formation) because the oil phase is liquid at room temperature and the interfacial tension between Vitamin E acetate oil and the water phase is lower than regular vegetable oils (e.g. sunflower oil).

On the other hand it is very much more difficult to emulsify carotenoids because of their low oil solubility and very low water solubility. Furthermore preventing recrystallization of the carotenoids requires that the ratio of cis vs. trans isomers will be finely tuned to achieve a stable system. Therefore emulsion containing carotenoids is in fact a different system than emulsion containing pure Vitamin E acetate. Finally emulsion droplet containing only oil soluble vitamin (such as Vitamin E acetate) cannot be used as colorant.

It is object of the present invention to put aqueous carotenoid emulsions at disposal which shows improved colour, preferably at high carotenoid concentrations, with improved bio-availability of the carotenoids. Additionally the emulsion has to be clear and transparent, meaning a very low turbidity. A further object of the present invention is to use exclusively naturally renewable raw material or naturally identically raw materials. Furthermore the emulsion has to be producible in an easy and effective way within only few steps.

It is further object of the present invention to put a carotenoid containing aqueous emulsions or a carotenoid containing powder as colorant at disposal which replace azo dyes and replicates the visual appearance of the end product coloured with azo dyes. Additionally is object of the invention to put aqueous carotenoid emulsions at disposal which does not contain any azo dyes but replicates the visual appearance of a product coloured with azo dyes.

These problems are solved by the emulsion of the present invention and the process for manufacturing the emulsion according to the invention.

In one embodiment the emulsion of the present invention is an aqueous transparent oil-in-water emulsion comprising an emulsified carotenoid in a concentration of 0.025-2000 ppm, preferably 0.025-300 ppm, 0.1-200 ppm, 0.1-100 ppm, 1-50 ppm, 1-30 ppm, 3-30 ppm, 3-20 ppm, more preferably 1-15 ppm, 3-15 ppm, especially 3-6 ppm. In one embodiment the concentration of the emulsified carotenoid is 5 ppm.

In one embodiment 1 ppm is 1 part per weight in 1 million parts per weight.

For the purpose of this invention an oil-in-water emulsion comprising an emulsified carotenoid means an emulsion of oil-droplets in water, whereby the carotenoid is present in the droplets and solved in the oil.

According to the present invention the term aqueous transparent oil-in-water emulsion means a clear emulsion with a low turbidity. The turbidity is measured with NTU which is nephelometric turbidity units that are measured with any standard turbidimeter, in one embodiment of the invention with HACH 2100AN Turbidimeter.

For the purpose of this invention a transparent emulsion is defined as an aqueous emulsion containing 5 ppm carotenoid with a NTU value below 35.

In one embodiment of the invention the aqueous emulsion containing 5 ppm has a turbidity value of 1-35 NTU, preferably 10-35 NTU, more preferably 15-30 NTU, especially 15-25 NTU, 15-20 NTU.

In a further embodiment of the invention the aqueous emulsion containing 5 ppm carotenoid has a turbidity value selected from the group consisting of: 15, 16, 17, 18, 19, 20, 21, 22, 23, 24 and 25 NTU.

In one alternative of the invention the carotenoid is selected from the group consisting of: cantaxanthin, astaxanthin, lutein, zeaxanthin, beta-zeacaroten, lycopene, apocarotenal, bixin, paprika olioresin, capsanthin and capsorubin, preferably apocarotenal, more preferably beta-apo-8-carotenal.

In an embodiment of the invention the carotenoid is beta-carotene.

In a further embodiment the oil-in-water emulsion of the invention is free from beta-carotene.

In the context of this invention, the characteristic “free from” means that the relevant compounds cannot be detected in the composition of the invention with the known state-of-the-art analytical methods for qualitatively and/or quantitatively detecting carotenoids, for example spectroscopy (UV/VIS), chromatography (HPLC). NMR and/or mass spectrometry.

All carotenoids can be natural or nature identical. Nature identical carotene is a synthetic carotene which has exactly the same chemical structure as natural carotene found in nature.

The emulsion of the present invention has in one alternative oil droplet size (using dynamic light scattering) of 50-400 nm, preferably 80-300 nm, more preferably 100-150 nm, especially 130-140 nm.

According to the invention the oil droplet size is the z-average size, measured in one embodiment using a Malvern zetasizer nano-s (serial nr, MAL500613).

The emulsion of the present invention has in one alternative polydispersity index (PDI) between 0.01 and 0.4, preferably between 0.01 and 0.3, more preferably between 0.01 and 0.2, especially between 0.01 and 0.1. Polydispersity index (PDI) defines the width of the oil droplet size distribution and is defined in ISP13321 part 8.

The emulsion of the present invention has in one alternative 70% of its oil droplets between 0 and 200 nm, preferably 80% of its oil droplets between 0 and 200 nm, more preferably 90% of its oil droplets between 0 and 200 nm, especially 95% of its oil droplets between 0 and 200 nm.

In one embodiment the present emulsion has a colour intensity value (E 1/1) of 140-280, preferably 140-250, 150-250, more preferably 150-230, 150-220, more preferably 160-230, 180-215, especially 205-215,

The colour intensity value of aqueous solutions (E 1/1) is defined as the absorbance of light at maximum absorbance (different for each carotenoid) going through 1 cm cuvette containing 1% carotenoid emulsion. If the colour intensity value (E 1/1) is measured at lower concentration than 1%, the measured value of the colour intensity has to be corrected with a dilution factor.

E1/1=(Aₘₐₓ×20)/(weight of sample(g))

In one embodiment of the present invention the carotenoid is melt and/or solved and/or isomerized from trans to cis in triacylglycerol oil, such as MCT oil (medium-chain triacylglycerol), olive oil, corn oil, sunflower oil, peanut oil, soy oil or other alternative vegetable oil, preferably MCI oil.

Accordingly the emulsion of the invention comprise in one embodiment triacylglycerol oil, selected from the group consisting of: MCT oil (medium-chain triacylglycerol), olive oil, sunflower oil, peanut oil, soy oil and vegetable oil, preferably MCT oil in a concentration of 0.1-150 ppm, preferably 1-75 ppm, more preferably 5-50 ppm, especially 15-30 ppm.

The triacylglycerol oil used in the present invention is in one embodiment an ester of glycerol where glycerol is esterified to a fatty acid where the fatty acid can have 4-22 carbon chain length and double bond on any of the carbon positions.

Preferably MCT oil is used, where the MCT is a ester of glycerol where glycerol is esterified to a fatty acid where the fatty acid are saturated and have 6-10 carbon chain length, preferably 8-10 carbon chain length.

The isomerization is carried out for an adequate time at a temperature of 100-200° C., 120-180° C., more preferably 130-160° C., till the trans-isomer content is between 30 and 100%, preferably 30 and 90%, more preferably 35 and 80%, especially 40 and 75%.

The isomerization can be carried out in the presence of an oil soluble antioxidant.

Accordingly in one embodiment of the invention the emulsion comprise a fat respectively an oil-soluble antioxidant in a concentration of 0.001-60 ppm, preferably 0.01-30 ppm, more preferably 0.1-20 ppm especially 0.5-12 ppm.

According to one embodiment of the present invention oil-soluble antioxidants are soluble in non-polar solvents and form a molecular dispersion.

Oil-soluble antioxidants according to the invention are selected from the group consisting of:
Ethoxyquin,
hindered phenolic antioxidants, such as t-butylhydroxytoluol, t-butylhydroxyaniso, t-butylhydroxyquinone;
Vitamin A, retinoic acid and its esters with C1-020 carbon chain length,
Vitamin D2 and D3,
alpha, beta, gamma, and delta Tocopherol or mixtures comprising at least two of the tocoperols;
alpha, beta, gamma, and delta Tocotrienols or mixtures comprising at least two of the tocotrienols;
natural extracts comprising at least one of the above compounds, phenolic diterpenes such as Carnosol, Carnosic add;
derivatives of cinnamic acid like 2-ethoxyethyl p-methoxycinnamate, ethylhexyl p-methoxycinnamate, 2-ethylhexyl 4-methoxycinnamate, methyl diisopropylcinnamate, isoamyl 4-methoxycinnamate, diethanolamin 4-methoxycinnamate and such as BASF products Tinogard TT, Tinogard HS, LC-gallates, Eugenol, Thymol, Organosolv-Lignin,
preferably alpha-tocopherol,
whereby the oil soluble antioxidant is in a concentration of 0.001-60 ppm, preferably 0.1-30 ppm, more preferably 1-20 ppm especially 6-12 ppm.

By using the process of the present invention the carotenoid shows a high stability solved in the triacylglycerol oil, so that no solid particles are within the droplets. The droplets are a mixture of carotenoid, optionally oil-soluble antioxidant and triacylglycerol oil.

In one embodiment the mixture of carotenoid, optionally oil-soluble antioxidant and triacylglycerol oil is a solution (meaning a molecular dispersion of the carotenoid, optionally oil-soluble antioxidant and triacylglycerol oil).

In one embodiment of the invention the carotene can be crystallized or partly crystallized.

In one alternative of the invention the emulsion comprises modified starch and/or at least one surface active protein selected from the group consisting of: gelatine, whey protein, whey protein isolate, Na-caseinate and other milk proteins, soy protein, potato protein; and/or natural polymers such as: lignin solfonate, pectin, Fenugreek gum and gum arabic, preferably modified starch, more preferably an octenyl succinate starch.

The modified starch and/or at least one surface active protein has a concentration of 0.5-600 ppm, preferably 2-300 ppm, more preferably 20-200 ppm, especially 60-120 ppm. The above mentioned polymers have a molecular weight MW-distribution of 10.000-2.000.000 g/mol, preferably 20.000-1.000.000 gμmol, more preferably 30.000-500.000 g/mol.

In an embodiment the emulsion of the invention is free from octenyl-succinic anhydride-modified gum acacia (gum acacia is also known as gum arabic).

In a further embodiment the emulsion comprises a carbohydrate selected from the group comprising: mono-, di- and oligosaccharides, glucose syrup, maltose and trehalose, preferably glucose syrup, maltose and trehalose, preferably from the group consisting of glucose-syrup, maltose and trehalose, more preferably glycose syrup and said carbohydrates have a DE (dextrose equivalent) between 20 and 50, preferably between 35 and 50 especially between 43 and 48. The saccharides contains glucose, fructose, galactose or mannose.

Said carbohydrate has a concentration of 0.5-10000 ppm, preferably 2-5000 ppm, more preferably 20-3000 ppm, especially 60-2000 ppm.

The emulsion of the present invention comprise in one alternative at east one water-soluble antioxidant selected from the group consisting of:
natural compounds that are active as antioxidants because they comprise a phenolic OH-group in their chemical structure: like hydroxy derivatives of cinnamic acid, e.g. hydroxycinnamic acids, hydroxycinnamates, which are a class of polyphenols having a C6-C3 skeleton, for example hydroxyhydrocinnamate;
caffeic acid, ferulic acid, tyrosol, hydroxytyrosol, cinnamic acid, chlorogenic acid, coumarin, coumarinic acid, sinapic acid, cinnamic acid, chicoric acid, and esters of any of these compounds with C1-020;
extracts of plants rich in at least one of the above compounds;
rosmarinic acid, hydroxytyrosol;
extracts from common spices. In one embodiment common spices are selected from the group comprising rosemary, lemon balm, oregano, thyme, peppermint, sage or similar plants comprising or being rich in at least one of the above compounds;
flavons, which are a class of natural compounds of which more than 5000 exist, used as antioxidants can be any of them as extracted from plants such as tea or any other plant that comprise or is rich in catechin or epicatechin or derivatives, whereby these compounds can be glycosylated with carbohydrates or esterified with fatty acids C1-C20 or gallic acid; extracts from plants such as tea, olives, pears, apples comprising or being rich in one or more of the above mentioned compounds;
sodium ascorbate, polyphenole, Teanova 80, glutathione, lipoic acid, catechin, punicalagin, xanthone, benzotropolones, preferably sodium ascorbate;
whereby the water soluble antioxidant is in a concentration of 0.001-60 ppm, preferably 0.1-30 ppm, more preferably 1-20 ppm, especially 6-12 ppm.

According to one embodiment of the present invention water-soluble antioxidants form a molecular dispersion in water.

Subject matter of the present invention is a process for manufacturing the emulsion comprising the following steps:
a) manufacturing a solution comprising modified starch and/or at least one surface active protein in a concentration of 0.7-70%, preferably 1-50%, more preferably 5-30%, especially 15-25%, at least one carbohydrate in a concentration of 0.001-80%, preferably 10-70%, more preferably 15-60%, especially 30-50% and water and mixing the components,
b) manufacturing a solution by mixing at least one carotenoid in a concentration of 0.1-15%, preferably 0.5-5%, more preferably 0.5-3%, especially 1-2%, in triacylglycerol oil in a concentration of 1-30%, preferably 3-20%, more preferably 3-15%, 3-10%, especially 3-5%, and melting the dispersed carotenoid, and optionally isomerizing from trans to cis said carotenoid, at a temperature of 100-200° C., 120-180° C., more preferably 130-160° C.,
c) introducing the solution of step b) into solution of step a),
d) performing at least one step of pre-emulsification by passing the mixture of step
c) through a rotator/stator mixer,
e) performing at least one step, preferably two steps of high pressure homogenization at a pressure of 300-2.000 bar,
f) spray drying the emulsion of step e) to obtain a powder,
g-1) manufacturing a emulsion by introducing the powder of step f) in water;
whereby the percent are weight percent referring to the emulsion of step c) to e) and the total amount of all used compounds is 100%.

A further subject matter is a process for manufacturing an oil-in-water emulsion comprising the steps a) to f) as mentioned above and manufacturing in step g-2) replacing step g-1) a emulsion by introducing the powder of step f) in water in a concentration of 5%-70%, preferably 10%-70%, 10%-60%, more preferably 20%-50%, whereby the percent are weight percent referring to the concentration of the powder according to step f) in water.

In one embodiment of the process of the invention optionally at least one water soluble antioxidant in a concentration of 0.001-10%, preferably 0.001-4%, more preferably 0.01-1.5%, especially 0.5-1.5 is added in step a).

In one embodiment of the process of the invention optionally at least one oil-soluble antioxidant in a concentration of 0.001-10%, preferably 0.001-5%, more preferably 0.01-2%, especially 0.1-1%, in triacylglycerol oil in a concentration of 1-30%, preferably 3—-20%, more preferably 3-15%, 3-10%, especially 3-5%, is added in step b).

In one alternative the process of the invention comprise one or more of the alternatives comprising:
mixing the carbohydrate into the emulsion in step c) or d)
adding the water soluble antioxidant after step c), d) or e),
adding the oil soluble antioxidant in step a), c) or d).

In one embodiment the pre-emulsification is carried out till the oil droplet size (z-average size) is 200 nm to 20 μm, preferably 250 nm to 10 μm, more preferably 300 nm to 5 μm.

The pre-emulsion is stable for at least 10 hours at 60° C. (with stirring), preferably 5 hours at 60° C. (with stirring), more preferably 3 hours at 60° C. (with stirring).

In one embodiment a two-stage homogenizer is used in the step of high pressure homogenization. The two-stage homogenizer consists of two pressure valves and therefore each pass though the machine consists of two stages. The first stage with 0-3000 bar, preferably 500-2000 bar, more preferably 650-1500 bar, especially 800-950 bar; and the second stage with 0-3000 bar, preferably 50-2000 bar, more preferably 50-1500 bar, especially 50-1000 bar.

In an alternative the emulsion is passed in one stage through a microfluidizer with 300-2000 bar, preferably 500-1500 bar, more preferably 500-1400 bar.

All steps in the process are conducted at temperatures between 0 and 500° C., preferably 20-200° C., more preferably 20-200° C. especially 50-200° C.

The step of high pressure homogenization is carried out in an alternative for at least 2, 3, 4, 5, 6, 7, 8, 9, 10, 15, 20, 30 or even more.

In one embodiment the process of the invention facilitates the manufacturing of stable emulsion without any surfactants.

In one embodiment the stable oil-in-water emulsion of the invention is free from other surfactants and/or emulsifiers than modified starch, preferably octenyl succinate starch. In other words, octenyl succinate starch is the only surfactant and/or emulsifier present in the emulsion of the invention.

Further subject matter of the present invention is the powder comprising a carotenoid according to step f).

The powder has in one alternative a residual moisture content of 0-15%, preferably 0.1-10%, more preferably 3-5%.

In one embodiment the powder produced according to the process of the invention comprises:
a modified starch and/or at least one surface active protein in a concentration of 0.7-70%, preferably 1-60%, more preferably 10-50%, especially 20-40%,
optionally at least one water soluble antioxidant in a concentration of 0.001-10%, preferably 0.001-4%, more preferably 0.001-2%, especially 1%,
at least one carbohydrate in a concentration of 0.001-80%, preferably 10-80%, more preferably 20-70%, especially 45%,
at least one carotenoid in a concentration of 0.5-20%, preferably 1-5%, more preferably 1-3%, especially 2-3%, preferably with a trans-isomer content between 30 and 100%,
optionally at least one oil-soluble antioxidant in a concentration of 0.001-10%, preferably 0.001-5%, more preferably 0.01-3%, especially 1%,
a triacylglycerol oil in a concentration of 1-50%, preferably 3-50%, 3-40%, more preferably 3-20%, 3-15%, 7-15%, especially 8%
and the total amount of all used compounds is 100%.

In one embodiment of the invention the powder is obtained by spray drying at inlet temperatures between 80° C. and 500° C., preferably 100-300° C., more preferably 100-200° C. and an outlet temperature of about 40-150° C., preferably 50-100° C., more preferably 50-90° C.

Another subject matter of the present invention is the (concentrated) emulsion comprising a carotenoid according to step g-2), manufactured by introducing the powder of step f) in water. This (concentrated) emulsion is a stock-emulsion which can be further diluted.

In one embodiment of the invention the aqueous, transparent emulsion, the concentrated stock-emulsion and/or the powder of the invention are used as colorant, preferably natural or naturally identical colorant. The aqueous emulsion, the concentrated stock-emulsion and/or the powder of the invention are used as colorant in beverages like soft drinks, flavoured water, fruit juices, punches or concentrated forms of these beverages but also alcoholic beverages and instant beverage powders.

In a further embodiment the aqueous emulsion, the concentrated stock-emulsion and/or the powder of the invention are used in food and/or feed. Typically the aqueous emulsion, the concentrated stock-emulsion and/or the powder of the invention are used in ice cream, cheese, milk product like milk drinks or yoghurt, soy milk and the like, confectionary products, gums, dessert, candies, puddings, jellies, instant pudding powder, but also in snacks, cookies, sauces, cereals, salad dressing, soups.

The aqueous solution, the concentrated stock-emulsion and/or the powder of the invention can also be used in pharmaceutical preparations, such as tablets or capsules, or cosmetic and dermal products.

In one embodiment the aqueous emulsion, the concentrated stock-emulsion and/or the powder of the invention are used instead of azo dyes and replace them. The products of the invention replace azo-dyes as food colorant, by replicating the visual appearance of the end product, meaning the final products have the same colour and transparency.

### EXAMPLES

### 1. Preparation Procedure

a) The modified starch, Na-ascorbic acid and the carbohydrate were dissolved in water having a temperature of 60° C.
b) Apocarotenal, including tocopherol was solved and isomerized in MCT-oil at 130-160° C. for 4 minutes.
c) The solution of step b) was mixed with the solution of step a)
d) The mixture of step c) was pre-emulsified in a lab scale rotator/stator mixer for 9 min.
e) The mixture was further emulsified by a high pressure microfluidyzer,
f) The emulsion was spray dried. The inlet temperature was 100 to 120° C. The outlet temperature was about 60° C.
g) A emulsion was manufactured by introducing the dry powder in water at a concentration of carotenoid of 5 ppm.

The powder had residual moisture content in a concentration of 5%.

In the following example the E1/1 value was measured at 460 nm maximum extinction, maximum extinction of 1% apocarotenal in solvent was 2640. Furthermore absorbance at 600 nm is an indication of turbidity.

2. Increasing Apocarotenal concentration

An emulsion according to example 1 was manufactured with the following compounds and concentration, whereby the mixture of step 1 e) was further emulsified by a high pressure microfluidyzer (1 pass at 1000 bar):

| | 2% | 3% | 4% | 5% |
|---|---|---|---|---|
| | apocarotenal | apocarotenal | apocarotenal | apocarotenal |
| Apocarotenal | 2 | 3 | 4 | 5 |
| (%) | | | | |
| MCT oil (%) | 8 | 12 | 16 | 20 |
| Glucose syrup | 48 | 38 | 34 | 30 |
| (%) | | | | |
| Modified starch | 40 | 40 | 40 | 40 |
| (%) | | | | |
| Na Ascorbate | 1 | 1 | 1 | 1 |
| (%) | | | | |
| Tocopherol (%) | 1 | 1 | 1 | 1 |
| Particle size | 126 | 142 | 148 | 155 |
| (nm) | | | | |
| Absorbance at | 0.016 | 0.03 | 0.05 | 0.06 |
| 600 nm | | | | |
| E1/1 value | 208 | 206 | 204 | 201 |
| Turbidity NTU | 17 | 31 | 33.7 | 34 |

### 3. Using Various Carbohydrates

A emulsion according to example 1 was manufactured with the following compounds and concentration, whereby the mixture of step 1 e) was further emulsified by a high pressure microfluidyzer (1 pass at 1000 bar):

| Trehalose as | | Glucose syrup as | | Glucidex 47 as | |
|---|---|---|---|---|---|
| carbohydrate | | carbohydrate | | carbohydrate | |
| Apocarotenal (%) | 2 | Apocarotenal (%) | 2 | Apocarotenal (%) | 2 |
| MCT oil (%) | 8 | MCT oil (%) | 8 | MCT oil (%) | 8 |
| Trehalose (%) | 48 | Glucose syrup (%) | 48 | Glucidex 47 (%) | 48 |
| Modified starch | 38 | Modified starch (%) | 38 | Modified starch | 38 |
| (%) | | | | (%) | |
| Na Ascorbate (%) | 1 | Na Ascorbate (%) | 1 | Na Ascorbate (%) | 1 |
| Tocopherol (%) | 1 | Tocopherol (%) | 1 | Tocopherol (%) | 1 |
| Particle size (nm) | 140 | Particle size (nm) | 130 | Particle size (nm) | 111 |
| Absorbance at | 0.022 | Absorbance at 600 nm | 0.02 | Absorbance at | 0.016 |
| 600 nm | | | | 600 nm | |
| E1/1 value | 208 | E1/1 value | 206 | E1/1 value | 207 |

### 4. Using Various Modified Starch Concentration

An emulsion according to example 1 was manufactured with the following compounds and concentration, whereby the mixture of step 1 e) was further emulsified by a high pressure microfluidyzer (1 pass at 800 bar):

| | 10% | 20% | |
|---|---|---|---|
| | starch | starch | 40% starch |
| Apocarotenal (%) | 2 | 2 | 2 |
| MCT oil (%) | 8 | 8 | 8 |
| Glucose syrup (%) | 75 | 65 | 45 |
| Modified starch | 10 | 20 | 40 |
| (%) | | | |
| Na Ascorbate (%) | 1 | 0 | 0 |
| Tocopherol (%) | 1 | 1 | 1 |
| Particle size (nm) | 113 | 134 | 131 |
| Absorbance at | 0.018 | 0.022 | 0.022 |
| 600 nm | | | |
| E1/1 value | 206 | 206 | 206 |
| Turbidity NTU | 23.9 | 19 | 18.7 |

### 5. Using Various Pressures

An emulsion according to example 1 was manufactured with the following compounds and concentration, whereby the mixture of step 1 e) was further emulsified by a high pressure microfluidyzer (1 pass at 600 or 1000 bar):

| | 600 bar | 800 bar | 1000 bar |
|---|---|---|---|
| Apocarotenal (%) | 2 | 2 | 2 |
| MCT oil (%) | 8 | 8 | 8 |
| Glucose syrup (%) | 48 | 48 | 48 |
| Modified starch (%) | 38 | 38 | 38 |
| Na Ascorbate (%) | 0 | 0 | 0 |
| Tocopherol (%) | 1 | 1 | 1 |
| Particle size (nm) | 150 | 131 | 123 |
| Absorbance at 600 nm | 0.036 | 0.022 | 0.019 |
| E1/1 value | 207 | 206 | 207 |

### 6. Passing 1-5 Times Through the Microfluidyzer

An emulsion according to example 1 was manufactured with the following compounds and concentration, whereby the mixture of step 1 e) was further emulsified by a high pressure microfluidyzer (at 1000 bar):

| | |
|---|---|
| Apocarotenal (%) | 1 |
| MCT oil (%) | 4 |
| Glucose syrup (%) | 49 |
| Modified starch (%) | 40 |
| Na Ascorbate (%) | 2 |
| Tocopherol (%) | 1 |
| Particle size 1 pass (nm) | 150 |
| Particle size 2 pass (nm) | 114 |
| Particle size 3 pass (nm) | 101 |
| Particle size 4 pass (nm) | 104 |
| Particle size 5 pass (nm) | 102 |

1. An aqueous transparent oil-in-water emulsion comprising an emulsified carotenoid in a concentration of 0.025-2000 ppm.

2. The emulsion according to claim 1 whereby the carotenoid is selected from the group consisting of: cantaxanthin, astaxanthin, lutein, zeaxanthin, beta-zeacaroten, lycopene, apocarotenal, bixin, paprika olioresin, capsanthin and capsorubin, preferably apocarotenal, more preferably beta-apo-8-carotenal.

3. The emulsion according to claim 1 with a NTU (Turbidity) value at a concentration of 5 ppm of carotenoid of 1-35 NTU.

4. The emulsion according to claim 1 with a colour intensity value (E 1/1) of 140-280, whereby the colour intensity value (E 1/1) is defined as the absorbance of light at maximum absorbance going through 1 cm cuvette containing 1% carotene dissolved in organic solvent.

5. The emulsion according to claim 1 with an oil droplets size measured with dynamic light scattering of 50-400 nm.

6. The emulsion according to claim 1 comprising a triacylglycerol oil, selected from the group consisting of: medium-chain triacylglycerol (MCT) oil, olive oil, sunflower oil, peanut oil, soy oil and vegetable oil, preferably MCT oil, in a concentration of 0.1-150 ppm.

7. The emulsion according to claim 1 comprising modified starch and/or at least one surface active protein, in a concentration of 0.5-600 ppm with a molecular weight MW-distribution of 10,000-2,000,000 g/mol.

8. The emulsion according to claim 1 comprising at least one carbohydrate selected from the group consisting of: mono-, di- and oligosaccharides, glucose-syrup, maltose and trehalose, said at least one carbohydrate having a DE between 20 and 50 in a concentration of 0.5-10000 ppm.

9. The emulsion according to claim 1 comprising at least one water soluble antioxidant selected from the group consisting of:
natural compounds that are active as antioxidants by comprising a phenolic OH-group in their chemical structure;
caffeic acid, ferulic acid, tyrosol, hydroxytyrosol, cinnamic acid, chlorogenic acid, coumarin, coumarinic acid, sinapic acid, cinnamic acid, chicoric acid, and esters of any of these compounds with C1-C20;
extracts of plants rich in at least one of the above compounds;
rosmarinic acid, hydroxytyrosol;
extracts from common spices;
flavons, preferably extracted from plants such as tea or any other plant that comprising catechin or epicatechin or derivatives;
extracts from plants such as tea, olives, pears, apples comprising one or more of the above mentioned compounds;
sodium ascorbate, polyphenole, Teanova 80, glutathione, lipoic acid, catechin, punicalagin, xanthone, benzotropolones, preferably sodium ascorbate, whereby the water soluble antioxidant is in a concentration of 0.001-60 ppm.

10. The emulsion according to claim 1 comprising an oil soluble antioxidant, selected from the group consisting of:
ethoxyquin,
hindered phenolic antioxidants, such as t-butylhydroxytoluol, t-butylhydroxyanisol, t-butylhydroxyquinone;
Vitamin A, retinoic acid and its esters with C1-C20;
Vitamin D2 and D3;
alpha, beta, gamma, and delta Tocopherol or mixtures comprising at least two of the tocopherols;
alpha, beta, gamma, and delta Tocotrienols or mixtures comprising at least two of the tocotrienols;
natural extracts comprising at least one of the above compounds,
phenolic diterpenes such as Carnosol, Carnosic acid;
derivatives of cinnamic acid like 2-ethoxyethyl p-methoxycinnamate, ethylhexyl p-methoxycinnamate, 2-ethylhexyl 4-methoxycinnamate, methyl diisopropylcinnamate, isoamyl 4-methoxycinnamate, diethanolamin 4-methoxycinnamate and
LC-gallates, Eugenol, Thymol,
whereby the oil soluble antioxidant is in a concentration of 0.001-60 ppm.

11. The emulsion according to claim 1 whereby the carotenoid has an isomerized, trans-isomer content between 30 and 100%.

12. A process for manufacturing the emulsion according to claim 1 comprising the following steps:
a) manufacturing a solution comprising modified starch and/or at least one surface active protein in a concentration of 0.7-70%, at least one sugar in a concentration of 0.001-80%, and water, and mixing the components,
b) manufacturing a solution by mixing at least one carotenoid in a concentration of 0.1-15%, in triacylglycerol oil in a concentration of 1-30% and melting the dispersed carotenoid at a temperature of 100-200° C.,
c) introducing the solution of step b) into the solution of step a),
d) performing at least one step of pre-emulsification by passing the mixture of step c) through a rotator/stator mixer,
e) performing at least one step of high pressure homogenization,
f) spray drying the emulsion of step e) to obtain a powder,
g-1) manufacturing an emulsion by introducing the powder of step f) in water;
whereby the percent are weight percent referring to the emulsion of step c) to e).

13. A process for manufacturing an oil-in-water emulsion comprising an emulsified carotenoid comprising the following steps:
a) manufacturing a solution comprising modified starch and/or at least one surface active protein in a concentration of 0.7-70%, at least one carbohydrate in a concentration of 0.001-80%, and water, and mixing the components,
b) manufacturing a solution by mixing at least one carotenoid in a concentration of 0.1-15% in triacylglycerol oil in a concentration of 1-30% and melting the dispersed carotenoid at a temperature of 100-200° C.,
c) introducing the solution of step b) into the solution of step a),
d) performing at least one step of pre-emulsification by passing the mixture of step c) through a rotator/stator mixer,
e) performing at least one step, preferably two steps of high pressure homogenization,
whereby the percent are weight percent referring to the emulsion of step c) to e);
f) spray drying the emulsion of step e) to obtain a powder, and
g-2) manufacturing an emulsion by introducing the powder of step f) in water in a concentration of 5%-70%, whereby the percent are weight percent referring to the concentration of the powder according to step f) in water.

14. The process of claim 12 comprising one or more of the alternatives comprising:
mixing the carbohydrate into the emulsion in step c) or d)
adding the water soluble antioxidant after step c), d) or e),
adding the oil soluble antioxidant in step a), c) or d).

15. A powder comprising a carotenoid made by the process according to step a) to f) of claim 12.

16. An oil-in-water emulsion comprising a carotenoid made by the process according to step a) to g-2) of claim 13.

17. A colorant, preferably a natural or nature identical colorant in food, feed, pharmaceutical preparations, cosmetic products, and/or dermal products comprising the powder of claim 15.

18. A method for replacing azo dyes comprising utilizing the powder of claim 15 as a colorant in food, feed, pharmaceutical preparations, cosmetic products and/or dermal products.

19. A colorant in food, feed, pharmaceutical preparations, cosmetic products, and/or dermal products, comprising the oil-in-water emulsion of claim 16.

20. A method for replacing azo dyes comprising utilizing the oil-in-water emulsion of claim 16 as a colorant in food, feed, pharmaceutical preparations, cosmetic products, and/or dermal products.
